(19) United States
(12) Patent Application Publication
(10) Pub. No.: US 2010/0100005 A1
(43) Pub. Date: Apr. 22, 2010

(54) MINIMALLY INVASIVE ALLERGY TESTING SYSTEM WITH COATED ALLERGENS

(75) Inventors: Jose Mir, Rochester, NY (US); Dennis Roland Zander, Penfield, NY (US)

Correspondence Address: HESLIN ROTHENBERG FARLEY & MESITI PC, 5 COLUMBIA CIRCLE, ALBANY, NY, 12203, US

(73) Assignee: INFOTONICS TECHNOLOGY CENTER, INC., Canandaigua, NY (US)

(21) Appl. No.: 12/373,385

(22) PCT Filed: Feb. 5, 2007
(86) PCT No.: PCT/US07/61604

§ 371 Date: Nov. 6, 2009

(30) Foreign Application Priority Data
Jul. 11, 2006 (US) PCT/US2006/026734
Jul. 11, 2006 (US) PCT/US2006/026774

(51) Int. Cl. A61B 5/00

(52) U.S. Cl. 600/556

(57) ABSTRACT

An allergy testing system has a microneedle array. The allergy testing system also has at least one coated allergen. The allergy testing system further has an activation system coupled to the microneedle array such that the at least one coated allergen is moved into contact with a subject as the microneedle array is moved from a resting position to a penetrating position. A method for determining a degree of reaction to one or more allergens by a patient in a minimally invasive manner is disclosed. One or more allergen coated microneedles is caused to penetrate into a skin of the patient. One or more images of at least one of the penetrations into the skin are captured. At least one of the captured images are analyzed to assess the degree of reaction to a specific allergen. Allergic reactivity data is output for at least one of the allergens.

### RELATED APPLICATIONS

This application claims priority to international application PCT/US2006/26774 filed on Jul. 11, 2006 and to international application PCT/US2006/26734 also filed on Jul. 11, 2006. In addition to containing some subject matter previously contained in those prior applications, this application also discloses new subject matter throughout the application.

### FIELD

The claimed invention generally relates to systems and methods for testing medical conditions and, more particularly, to systems used to determine a degree of reaction to one or more allergens by a subject in a minimally invasive manner.

### BACKGROUND

It is estimated that allergies affect over 50 million people in the United States and result in billions of dollars of medical costs and lost productivity in industrialized countries. On average, primary care physicians see one allergy patient per day. Some of these allergy patients get referred to allergy specialists but many more are temporarily treated for their symptoms or misdiagnosed as having unrelated respiratory problems. Undiagnosed and untreated allergies potentially increase the patient's probability of increased sensitization or progressive stages of allergy known in Europe as the “allergic march”. Adding to these concerns, industrialized Countries such as Great Britain and the US are experiencing unprecedented growth in allergy sensitivity, doubling or tripling in the last three decades. Allergic rhinitis today ranks as the world's most prevalent immunological affliction and chronic health problem while asthma is the most common illness diagnosed during U.S. hospital admissions. Although allergic rhinitis has unpleasant symptoms such as rhinorrhea and congestion, other more severe allergic reactions to insect stings, nut/foods, drugs, and occupational substances can result in serious medical conditions such as anaphylaxis and death.

The dangers that these allergies represent are undeniable. However, while the number of people actually tested for allergies is on the rise, far more people never get tested and run the risk that undiagnosed allergies are present. Unfortunately, many allergies are diagnosed only after serious symptoms are noticed and before precautions can be taken to avoid the allergen or prevent further sensitization, resulting in hospitalization, loss of productivity from sick days, and even death.

Physicians prescribe allergy testing because early diagnosis can help patients take preventive measures such as managing their environment, avoiding certain drugs or foods, availing themselves of medication, immunotherapy, or self-administered epinephrine. Allergy tests such as hypersensitivity skin testing have been used for decades because of their sensitivity, reliability, and relative immediacy of results. The widely-used Skin Prick (percutaneous) Test involves the manual application of as many as 40 standardized allergen extracts in a labeled array pattern on the patient's back or forearm, subsequently pricking the region with a needle to allow diffusion of allergens into the patient's epidermal tissue. The intradermal test (intracutaneous) is somewhat more invasive and labor intensive since it involves injecting small amounts of allergens into the patient's skin. The procedure, while more sensitive than percutaneous tests, has a somewhat lower predictive value. Intracutaneous testing is most often used to diagnose insect venom and drug allergies. Due to the nature of these processes, large areas of the subject's skin tend to be affected.

In both of these types of hypersensitivity skin testing, reactions to the allergens are assessed after a time period of approximately 20 minutes based on the area and coloration of wheals or erythemas produced by each allergen. Sensitivity is diagnosed and recorded using semi-quantitative guidelines and controls such as saline and histamine. Although the procedure is not highly painful and produces only temporary tenderness, itching, or swelling, it can lead to significant stress and anxiety in many patients, especially children. In addition to psychological effects the test may have on some patients, there may be other related problems: 1) Degree of invasiveness and skin surface area covered; 2) Time and manual labor needed to label, dispense, and prick regions with allergens; 3) Lack of an automated standardized protocol to capture the data and store as a digital record.

Blood testing provides an invasive, yet possibly more convenient method of allergy testing. Unfortunately, blood testing does not surpass the sensitivity, specificity, and predictive value of the skin test. Blood test results are often dependent upon the laboratory which is performing the test. Furthermore, blood testing for allergies is also a more expensive option.

The alarming prevalence of allergies and their rapid growth indicate that the demand for allergists and immunologists will increase in the years to come. Unfortunately, American Academy of Allergy, Asthma, & Immunology (AAAAI) surveys indicate that the number of graduating allergists may not be sufficient to address future health needs. Furthermore, demographic studies of the approximately 4000 allergists in the U.S. show that a disproportionately high number of core allergists will likely retire in the near future, further exacerbating the problem. These trends indicate not only that the number of allergists per capita will decline but, more importantly, that the number of allergists per allergy patient will decline at an even faster rate. Not surprisingly, allergy practices, especially those run by younger physicians, are experiencing rapid growth in the number of patient visits.

These trends point to a compelling need for less invasive, more quantitatively productive allergy tests with shorter procedure times. Less invasive, shorter, lower cost procedures are also likely to reduce patient fear and reduce barriers associated with current procedures. Improved testing might also promote early or preventative allergy screening mitigating “allergic march”, sensitization, or the cost and trauma of serious reactions.

Therefore, there is a need for a minimally invasive allergy testing system which does not need to cover large areas of a patient's skin, which can be automated to a large extent, which can be correlated to existing skin testing data, which offers more than a snapshot in time of an allergic reaction, which is economical, and which is easy to use and manufacture.

### SUMMARY

An allergy testing system is disclosed. The allergy testing system has a microneedle array. The allergy testing system also has at least one coated allergen. The allergy testing system further has an activation system coupled to the microneedle array such that the at least one coated allergen is moved into contact with a subject as the microneedle array is moved from a resting position to a penetrating position.

Another allergy testing system is disclosed. The allergy testing system has an attachment band having a test frame, wherein the test frame defines an opening in the attachment band. The allergy testing system also has a package, for interfacing with the test frame. The package has a microneedle array and coated allergens. The allergy testing system also has an imaging system for interfacing with the test frame. The allergy testing system further has an analyzer coupled to the imaging system.

A method for determining a degree of reaction to one or more allergens by a patient in a minimally invasive manner is disclosed. One or more allergen coated microneedles is caused to penetrate into a skin of the patient. One or more images of at least one of the penetrations into the skin are captured. At least one of the captured images are analyzed to assess the degree of reaction to a specific allergen. Allergic reactivity data is output for at least one of the allergens.

Another method for determining a degree of reaction to one or more allergens by a patient in a minimally invasive manner is disclosed. One or more coated allergens on a sheet are wetted to dissolve the coated allergens. One or more microneedles are caused to penetrate into a skin of the patient. Each of the penetrations into the skin are exposed with dissolved coated allergens. One or more images of each of the penetrations into the skin are captured. At least one of the captured images are analyzed to assess the degree of reaction to the specific allergen. Allergic reactivity data is output for at least one of the allergens.

The claimed invention provides a system and method to minimize the invasiveness of allergy testing, degree and area of reaction, testing time, general discomfort, and risk of infection. Another advantage of the claimed invention is that it enables a much smaller test area footprint when compared to prior testing devices. The much smaller footprint also simplifies and expedites the allergy testing process for a medical staff. The claimed invention uses dried allergens that are used only once, hence avoiding contamination and aging compared to the current method of liquid storage. The allergen dispensing process may also be automated in some embodiments, allowing for automated and quantified allergy reactivity data readout, thereby reducing uncertainty and subjectivity. A further advantage possible with automated embodiments is the ability to capture continuous or nearly continuous visual images of an allergy test site. This allows scientist and medical personnel a chance to study the time rate of change for certain allergic reactions, and better understand a patient's reaction and sensitivity. Overall, the minimally-invasive allergy testing system enables a relatively fast allergy test cycle time, lowers the cost of such testing, and significantly reduces the chance for errors.

### BRIEF DESCRIPTION OF THE DRAWINGS

FIGS. 1-2L schematically illustrate embodiments of an allergy testing system.

FIGS. 3A & 3B to FIGS. 8A & 8B schematically illustrate side views (A) and top views (B), respectively, of embodiments of microneedles for use in an allergy testing system.

FIG. 9 schematically illustrates a side view of a corrugated microneedle embodiment for use in an allergy testing system.

FIG. 10A schematically illustrates an exploded perspective view of an embodiment of an allergy testing system.

FIG. 10B schematically illustrates an assembled, cross-sectional view of the allergy testing system embodiment of FIG. 10A.

FIG. 11A schematically illustrates an exploded perspective view of an embodiment of an allergy testing system.

FIG. 11B schematically illustrates an assembled, cross-sectional view of the allergy testing system embodiment of FIG. 11A.

FIGS. 12A-12C schematically illustrate one possible method of applying allergens to a subject using an embodiment of an allergy testing system.

FIGS. 12D1-12D3 schematically illustrate different embodiments of gathering and analyzing allergy test data after the allergens have been applied by the allergy testing system in FIGS. 12A-12C.

FIG. 13 schematically illustrates an embodiment of an allergy testing system.

FIGS. 14A-14B schematically illustrate a side view of an embodiment of an allergy testing system.

FIGS. 15A-15B schematically illustrate a top view of an embodiment of an allergy testing system.

It will be appreciated that for purposes of clarity and where deemed appropriate, reference numerals have been repeated in the figures to indicate corresponding features, and that the various elements in the drawings have not necessarily been drawn to scale in order to better show the features.

### DETAILED DESCRIPTION

FIG. 1 schematically illustrates an embodiment of a minimally invasive allergy testing system 20. The minimally invasive allergy testing system 20 has a microneedle array 22 and at least one coated allergen 24. The microneedle array 22 has at least one microneedle, and preferably a plurality of microneedles which may be spaced in a linear array, a two-dimensional array, or any other spacing desired. The microneedles may have a height of about 50-1000 microns and a tip dimension ranging from submicron to about 80 microns in order to penetrate a subject's skin, although other embodiments may have other dimensions. (Skin not illustrated in this view.) The microneedle 22 array may be manufactured out of a number of different substances, for example, silicon, glass, metal, quartz, or plastic. Due to its attractive micromachining properties, silicon may be etched using chemical and reactive ion etching processes to fabricate the microneedles, although other materials and manufacturing processes can be used.

The microneedle array 22 and the one or more coated allergens 24 may be aligned relative to each other by virtue of the coated allergens 24 being coated directly onto the microneedle array 22, or by virtue of the coated allergens 24 being coated on a another structure, such as a sheet, film, or label held in alignment relative to the microneedle array 22. The minimally invasive allergy testing system 20 also has an activation system 26 which may be directly or indirectly coupled to the microneedle array 22. The activation system 26 causes a skin of a test subject to be pricked, while allowing the coated allergens 24 to dissolve within the subject's skin to initiate a controlled allergic reaction. In some embodiments, as will be discussed further in this disclosure, the coated allergens 24 may be dissolved prior to the pricking of the subject's skin by wetting the coated allergens 24 with a solvent.

There is a great degree of flexibility in configuring the activation system 26. In some embodiments, the activation system 26 can be a mechanical plunger or other mechanical system, which is pressed by a medical professional, or even the test subject themselves. In other embodiments, the activation system 26 can be a spring-loaded release which allows a predictable force to be applied to the microneedle array 22 as it pricks the subject's skin. Further embodiments of an allergy testing system 20 may have an activation system 26 which is an electro-mechanical system, such as a solenoid, motor, or a micromechanical actuator.

The microneedle array 22, the coated allergens 24, and/or the activation system 26 may be separate components of the allergy testing system 20. In various embodiments, the microneedle array 22 and the coated allergens 24 may be removeably packaged from the activation system 26. This removeability allows for designs and embodiments with simple replacement of the microneedles and coated allergens which are typically only used once.

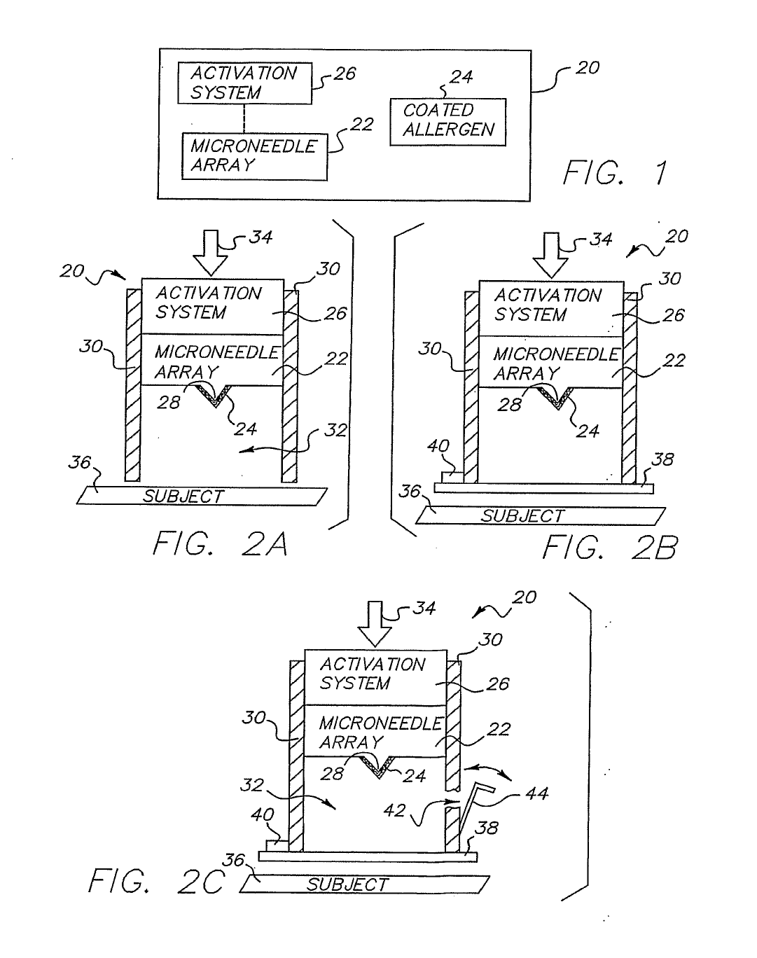

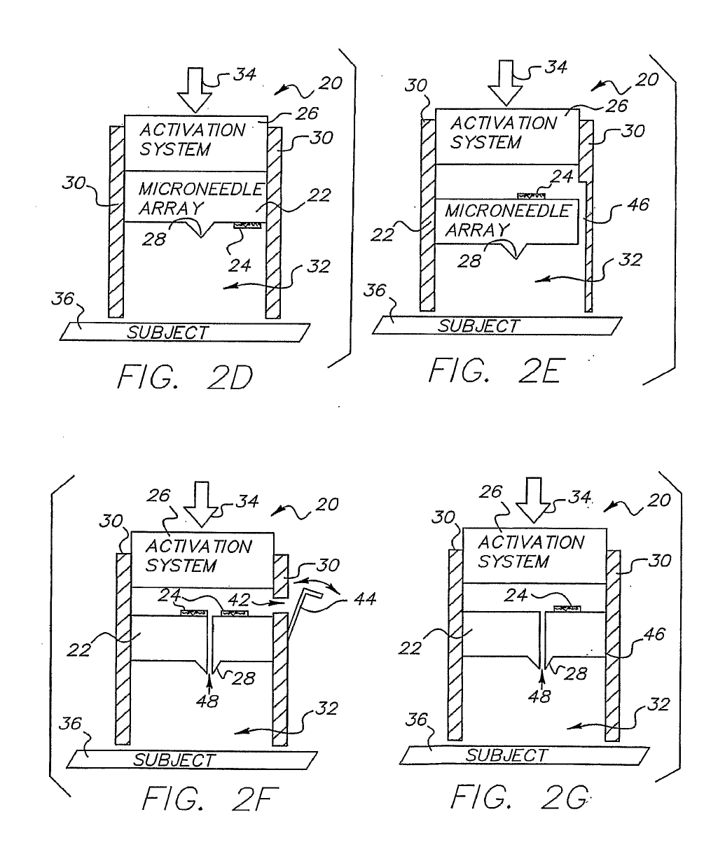

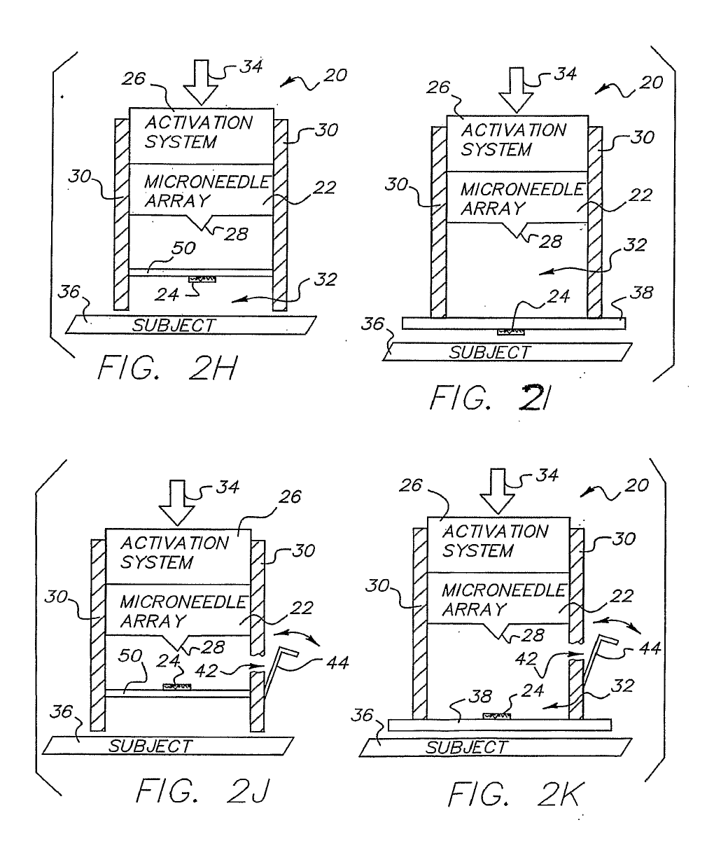

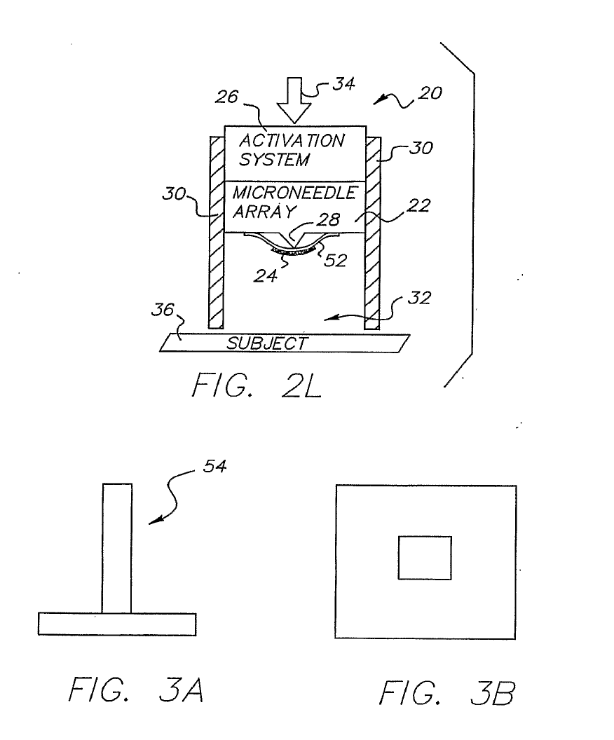

FIG. 2A illustrates another embodiment of an allergy testing system 20. This allergy testing system 20 has the microneedle array 22 directly coupled to the activation system 26. The microneedle array 22 may still be removeably coupled to the activation system 26 in some embodiments. For simplicity, the microneedle array 22 is schematically illustrated in this and other embodiments as only having a single needle 28. It should be understood that any number of microneedles 28 may be present on the microneedle array 22, depending on size of the microneedle array 22 and the number of allergy test sites desired. The activation system 26 and the microneedle array 22 are moveably supported by a package 30. The package 30 defines at least one orifice 32 within which one or more of the microneedles 28 may be moved. In this embodiment, the coated allergen 24 is coated directly onto the microneedle 28 of the microneedle array 22. The coated allergen 24 may be coated onto the microneedle 28 as a pure extract, or can be dissolved into a solution containing film forming agents such as glycerol, biocompatible water soluble polymer, or other addenda. These film forming agents operate as binders which assist the coated allergen 24 in adhering to the microneedle 28. The film forming agents may also be used to improve coatability and wetting during manufacturing. In other embodiments, a binder layer can be coated directly onto the microneedle 28 prior to coating with allergens. In the orientation of FIG. 2A, the activation system 28 will engage downwards 34 to push the microneedles 28 of the microneedle array 22 into a subject's skin 36. The activation system 26 may allow the coated microneedles 28 to penetrate the skin 36 for a period of time sufficient for the coated allergen 24 to dissolve in the fluids found within the subject's skin 36 and/or on the subject's skin 36, thereby allowing the allergen to enter the skin 36. This time is variable and depends on the solubility of the allergen and any addenda. Useful times for some embodiments may be in the range of 0.3-5.0 seconds, however other embodiments may require shorter or longer times.

The allergy testing system embodied in FIG. 2A could also be pre-treated with a solvent, such as water, just prior to the administration of the allergy test to assist the coated allergens 24 in dissolving, or if it is simply desired to have the coated allergens 24 in dissolved form prior to penetrating the subject's skin. In this case, access for wetting the coated allergens 24 may be provided through the orifices 32 in the package 30.

FIG. 2B schematically illustrates another embodiment of an allergy testing system 20 which is similar to the allergy testing system 20 of FIG. 2A, with the addition of a sealing film 38 to cover the microneedle array 22 and the coated allergens 24. The sealing film 38 may be employed over at least a portion of the package 30 to serve as a sterile layer protecting the sterile microneedles 28 and the coated allergens 24. The sealing film 38 may be removed before use or left on the patient's skin 36. If left behind on the skin 36, the sealing film 38 may have a human readable code or a machine readable code 40, such as a bar code, or other identification marks 40. These identifying marks 40 can also be used for orientation in the analysis stage to be discussed later in this specification. The identifying marks 40 can also identify the various allergens being used in one or more locations.

FIG. 2C schematically illustrates another embodiment of an allergy testing system 20 which is similar to the embodiment of FIG. 2B. However, the embodiment of FIG. 2C has an access hole 42 defined by the package 30 which may be used to provide access for a solvent to be applied to the coated allergen 24 prior to administration of the allergy test. Depending on the embodiment, there could be one or more access holes 42. It may be desirable to assist the coated allergens 24 in dissolving, or it may simply be desired to have the coated allergens 24 in dissolved form prior to penetrating the subject's skin. A sealing flap 44 may be provided in some embodiments to cover the access hole 42 to allow a solvent to be agitated within the orifice 32 without leaking prior to administration of an allergy test or removal of the sealing film 38.

FIG. 2D schematically illustrates another embodiment of an allergy testing system 20. This allergy testing system 20 has the microneedle array 22 coupled to the activation system 26. The activation system 26 and the microneedle array 22 are moveably supported by a package 30. The package 30 defines at least one orifice 32 within which the microneedle 28 may be moved. In this embodiment, the coated allergen 24 is coated directly onto the microneedle array 22, but not in the area of the microneedle 28. The coated allergen 24 may be coated onto the microneedle array 22 as a pure extract, or can be dissolved into a solution containing film forming agents such as glycerol, biocompatible water soluble polymer, or other addenda. These film forming agents operate as binders which assist the coated allergen 24 in adhering to the microneedle array 22. In other embodiments, a binder layer can be coated directly onto the microneedle array 22 prior to coating with allergens.

Prior to activating the allergy testing system 20, the coated allergens 24 may be wetted with a solvent via the orifice 32. These wetted allergens may then coat the microneedle 28. Once the coated allergens 24 have been wetted, the activation system 26 may be engaged downwards 34 to push the microneedles 28 of the microneedle array 22 into a subject's skin 36. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

FIG. 2E schematically illustrates another embodiment of an allergy testing system 20. This allergy testing system 20 may be configured to have the microneedle array 22 coupled to the activation system 26, however, for simplicity, the activation system is illustrated in this embodiment separate from the microneedle array 22. The activation system 26 and the microneedle array 22 are moveably supported by a package 30. The package 30 defines at least one orifice 32 within which the microneedle 28 may be moved. In this embodiment, the coated allergen 24 is coated directly onto the microneedle array 22, but not on the side of the microneedle array 22 having the microneedle 28. Instead, in this embodiment, the coated allergen 24 is coupled to a second side of the microneedle array 22. The coated allergen 24 may be coated onto the microneedle array 22 as a pure extract, or can be dissolved into a solution containing film forming agents such as glycerol, biocompatible water soluble polymer, or other addenda. These film forming agents operate as binders which assist the coated allergen 24 in adhering to the microneedle array 22. In other embodiments, a binder layer can be coated directly onto the microneedle array 22 prior to coating with allergens.

The package 30 defines a channel 46 which fluidically couples the microneedle 28 side of the microneedle array 22 to the side of the microneedle array 22 with the coated allergen 24. In other embodiments, the channel 46 may be provided by the microneedle array 22 or a combination of the microneedle array 22 and the package 30. Prior to activating the allergy testing system 20, the coated allergens 24 may be wetted with a solvent via the orifice 32 and the channel 46. After wetting, when the activation system 26 is engaged downwards 34 to push the microneedles 28 of the microneedle array 22 towards a subject's skin 36, the motion of the activation system 26 against the microneedle array 22 may squeeze the wetted allergens 24 out the channel 46 and onto the microneedle 28 as it penetrate the skin 36 for a desired period of time. Useful times for some embodiments may be in the range of 0.3-5.0 seconds, however other embodiments may require shorter or longer times.

FIG. 2F schematically illustrates another embodiment of an allergy testing system 20. This allergy testing system 20 may be configured to have the microneedle array 22 coupled to the activation system 26, however, for simplicity, the activation system 26 is illustrated in this embodiment separate from the microneedle array 22. The activation system 26 and the microneedle array 22 are moveably supported by a package 30. The package 30 defines at least one orifice 32 within which the microneedle 28 may be moved. In this embodiment, the coated allergen 24 is coated directly onto the microneedle array 22, but not on the side of the microneedle array 22 having the microneedle 28. Instead, in this embodiment, the coated allergen 24 is coupled to a second side of the microneedle array 22. The coated allergen 24 may be coated onto the microneedle array 22 as a pure extract, or can be dissolved into a solution containing film forming agents such as glycerol, biocompatible water soluble polymer, or other addenda. These film forming agents operate as binders which assist the coated allergen 24 in adhering to the microneedle array 22. In other embodiments, a binder layer can be coated directly onto the microneedle array 22 prior to coating with allergens.

In this embodiment, the microneedle 28 defines a channel 48 which fluidically couples the microneedle 28 side of the microneedle array 22 to the side of the microneedle array with the coated allergen 24. Prior to activating the allergy testing system 20, the coated allergens 24 may be wetted with a solvent via an access hole 42. As mentioned above, the access hole 42 may be provided with a plug 44 for removeably sealing the access hole 42. After wetting, when the activation system 26 is engaged downwards 34 to push the microneedles 28 of the microneedle array 22 towards a subject's skin 36, the motion of the activation system 26 against the microneedle array 22 may squeeze the wetted allergens out the channel 48, through the microneedle 28, and into the skin 36 as the microneedle 28 penetrates the skin 36 for a desired period of time. Useful times for some embodiments may be in the range of 0.3-5.0 seconds, however other embodiments may require shorter or longer times.

FIG. 2G schematically illustrates another embodiment of an allergy testing system 20. This allergy testing system 20 may be configured to have the microneedle array 22 coupled to the activation system 26, however, for simplicity, the activation system 26 is illustrated in this embodiment separate from the microneedle array 22. The activation system 26 and the microneedle array 22 are moveably supported by a package 30. The package 30 defines at least one orifice 32 within which the microneedle 28 may be moved. In this embodiment, the coated allergen 24 is coated directly onto the microneedle array 22, but not on the side of the microneedle array 22 having the microneedle 28. Instead, in this embodiment, the coated allergen 24 is coupled to a second side of the microneedle array 22. The coated allergen 24 may be coated onto the microneedle array 22 as a pure extract, or can be dissolved into a solution containing film forming agents such as glycerol, biocompatible water soluble polymer, or other addenda. These film forming agents operate as binders which assist the coated allergen 24 in adhering to the microneedle array 22. In other embodiments, a binder layer can be coated directly onto the microneedle array 22 prior to coating with allergens.

In this embodiment, the microneedle 28 defines a channel 48 which fluidically couples the microneedle 28 side of the microneedle array 22 to the side of the microneedle array with the coated allergen 24. Prior to activating the allergy testing system 20, the coated allergens 24 may be wetted with a solvent via the orifice 32 and the channel 48. After wetting, when the activation system 26 is engaged downwards 34 to push the microneedles 28 of the microneedle array 22 towards a subject's skin 36, the motion of the activation system 26 against the microneedle array 22 may squeeze the wetted allergens out the channel 48, through the microneedle 28, and into the skin 36 as the microneedle 28 penetrates the skin 36 for a desired period of time. Useful times for some embodiments may be in the range of 0.3-5.0 seconds, however other embodiments may require shorter or longer times.

FIG. 2H schematically illustrates another embodiment of an allergy testing system 20. This allergy testing system 20 has the microneedle array 22 coupled to the activation system 26. The activation system 26 and the microneedle array 22 are moveably supported by a package 30. The package 30 defines at least one orifice 32 within which the microneedle 28 may be moved. The allergy testing system 20 also has a sheet 50 coupled to the package 30. One side of the sheet 50 is disposed towards the microneedle array 22 and the other side of the sheet 50 is disposed away from the microneedle array 22. In this embodiment, the coated allergen 24 is coated directly onto the side of the sheet 50 facing away from the microneedle array 22. The coated allergen 24 may be coated onto the sheet 50 as a pure extract, or can be dissolved into a solution containing film forming agents such as glycerol, biocompatible water soluble polymer, or other addenda. These film forming agents operate as binders which assist the coated allergen 24 in adhering to the sheet 50. Sheet 50 may even be selected for its properties in allowing the allergens to coat properly thereon without the need for a binder.

Prior to activating the allergy testing system 20, the coated allergens 24 may be wetted with a solvent via the orifice 32. The activation system 26 may then be engaged downwards 34 to push the microneedles 28 of the microneedle array 22 through the sheet 50, supporting the now dissolved allergens 24, and into a subject's skin 36. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

FIG. 2I schematically illustrates another embodiment of an allergy testing system 20. This allergy testing system 20 has the microneedle array 22 coupled to the activation system 26. The activation system 26 and the microneedle array 22 are moveably supported by a package 30. The package 30 defines at least one orifice 32 within which the microneedle 28 may be moved. The allergy testing system 20 also has a sealing film 38 coupled to the package 30 and covering at least a portion of the orifice 32. One side of the sealing film 38 is disposed towards the microneedle array 22 and the other side of the sealing film 38 is disposed away from the microneedle array 22. In this embodiment, the coated allergen 24 is coated directly onto the side of the sealing film 38 facing away from the microneedle array 22. The coated allergen 24 may be coated onto the sealing film 38 as a pure extract, or can be dissolved into a solution containing film forming agents such as glycerol, biocompatible water soluble polymer, or other addenda. These film forming agents operate as binders which assist the coated allergen 24 in adhering to the sealing film 38. Sealing film 38 may even be selected for its properties in allowing the allergens to coat properly thereon without the need for a binder.

Prior to activating the allergy testing system 20, the coated allergens 24 may be wetted with a solvent. The sealing film and wetted allergens may then be placed against the subject's skin 36, and the activation system 26 may then be engaged downwards 34 to push the microneedles 28 of the microneedle array 22 through the sealing film 38, supporting the now dissolved allergens 24, and into a subject's skin 36. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

FIG. 2J schematically illustrates another embodiment of an allergy testing system 20. This allergy testing system 20 has the microneedle array 22 coupled to the activation system 26. The activation system 26 and the microneedle array 22 are moveably supported by a package 30. The allergy testing system 20 also has a sheet 50 coupled to the package 30. One side of the sheet 50 is disposed towards the microneedle array 22 and the other side of the sheet 50 is disposed away from the microneedle array 22. In this embodiment, the coated allergen 24 is coated directly onto the side of the sheet 50 facing towards the microneedle array 22. The coated allergen 24 may be coated onto the sheet 50 as a pure extract, or can be dissolved into a solution containing film forming agents such as glycerol, biocompatible water soluble polymer, or other addenda. These film forming agents operate as binders which assist the coated allergen 24 in adhering to the sheet 50. Sheet 50 may even be selected for its properties in allowing the allergens to coat properly thereon without the need for a binder.

Prior to activating the allergy testing system 20, the coated allergens 24 may be wetted with a solvent via an access hole 42 defined by the package 30. A plug 44 may be provided to removeably seal the access hole 42. The activation system 26 may then be engaged downwards 34 to push the microneedles 28 of the microneedle array 22 through the dissolved allergens 24, supported by the sheet 50, and into a subject's skin 36. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

FIG. 2K schematically illustrates another embodiment of an allergy testing system 20. This allergy testing system 20 has the microneedle array 22 coupled to the activation system 26. The activation system 26 and the microneedle array 22 are moveably supported by a package 30. The package 30 defines at least one orifice 32 within which the microneedle 28 may be moved. The allergy testing system 20 also has a sealing film 38 coupled to the package 30 and covering at least a portion of the orifice 32. One side of the sealing film 38 is disposed towards the microneedle array 22 and the other side of the sealing film 38 is disposed away from the microneedle array 22. In this embodiment, the coated allergen 24 is coated directly onto the side of the sealing film 38 facing towards the microneedle array 22. The coated allergen 24 may be coated onto the sealing film 38 as a pure extract, or can be dissolved into a solution containing film forming agents such as glycerol, biocompatible water soluble polymer, or other addenda. These film forming agents operate as binders which assist the coated allergen 24 in adhering to the sealing film 38. Sealing film 38 may even be selected for its properties in allowing the allergens to coat properly thereon without the need for a binder.

Prior to activating the allergy testing system 20, the coated allergens 24 may be wetted with a solvent via an access hole 42 defined by the package 30. A plug 44 may also be provided to removeably seal the access hole 42. The sealing film may then be placed against the subject's skin 36, and the activation system 26 may then be engaged downwards 34 to push the microneedles 28 of the microneedle array 22 through the dissolved allergens 24 supported by the sealing film 38, and into a subject's skin 36. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

FIG. 2L schematically illustrates another embodiment of an allergy testing system 20. This allergy testing system 20 has the microneedle array 22 coupled to the activation system 26. The activation system 26 and the microneedle array 22 are moveably supported by a package 30. The allergy testing system 20 also has a sheet 52 coupled to the microneedle array 22. One side of the sheet 52 is disposed towards the microneedle array 22 and the other side of the sheet 52 is disposed away from the microneedle array 22. In this embodiment, the coated allergen 24 is coated directly onto the side of the sheet 52 facing away from the microneedle array 22. The coated allergen 24 may be coated onto the sheet 52 as a pure extract, or can be dissolved into a solution containing film forming agents such as glycerol, biocompatible water soluble polymer, or other addenda. These film forming agents operate as binders which assist the coated allergen 24 in adhering to the sheet 52. Sheet 52 may even be selected for its properties in allowing the allergens to coat properly thereon without the need for a binder.

Prior to activating the allergy testing system 20, the coated allergens 24 may be wetted with a solvent via the orifice 32. The activation system 26 may then be engaged downwards 34 to push the microneedles 28 and the sheet 50, supporting the now dissolved allergens 24, and into contact with a subject's skin 36. When the sheet 52 reaches the skin 36, the microneedle 28 will be able to exert pressure on the sheet 52, piercing it, and penetrating the skin along with the dissolved allergens 24 which were on the sheet. The activation system 26 may allow the microneedles 28 with allergens 24 to penetrate the skin 36 for a desired period of time. Useful times for some embodiments may be in the range of 0.3-5.0 seconds, however other embodiments may require shorter or longer times.

The microneedles in the microneedle array 22 may have a variety of geometries. FIGS. 3A and 3B schematically illustrate an embodiment of a microneedle 54 with a substantially square or rectangular cross-section in a side view and a corresponding top view, respectively.

FIGS. 4A and 4B schematically illustrate an embodiment of a microneedle 56 with a substantially circular cross-section in a side view and a corresponding top view, respectively. The microneedle embodiment shown in FIG. 4A illustrates another variation possible with microneedle design. The microneedle 56 has a wedged top. Although other embodiments are not shown wedged, they could be modified in further embodiments to have a wedge-shaped top.

FIGS. 5A and 5B schematically illustrate an embodiment of a microneedle 58 with a substantially triangular cross-section in a side view and a corresponding top view, respectively.

FIGS. 6A and 6B schematically illustrate an embodiment of a microneedle 60 with a substantially square or rectangular cross-section in a side view and a corresponding top view, respectively. This microneedle, however, is a hollow microneedle, having a channel formed within the needle for the passage of allergens.

FIGS. 7A and 7B schematically illustrate an embodiment of a microneedle 62 with a substantially square or rectangular cross-section in a side view and a corresponding top view, respectively. This microneedle, however, is a grooved microneedle, having a channel formed on the side of the needle for the passage of allergens.

FIGS. 8A and 8B schematically illustrate an embodiment of a microneedle 64 with a changing cross-sectional area that tapers to a point.

Any of the features of the microneedles in the embodiments of FIGS. 3A-8B may be combined with one another, and cross-sectional shapes may be varied. For example, a microneedle could be both hollowed and grooved at the same time. As mentioned before, the microneedles may have a height of about 50-1000 microns and a tip dimension from submicron to about 80 microns in order to penetrate a subject's skin, although other embodiments may have other dimensions. (Skin not illustrated in this view.) The microneedle array may be manufactured out of a number of different substances, for example, silicon, glass, metal, or plastic. Due to its attractive micromachining properties, silicon may be anisotropically etched using chemical and reactive ion etching processes to fabricate the microneedles, although other materials and manufacturing processes can be used.

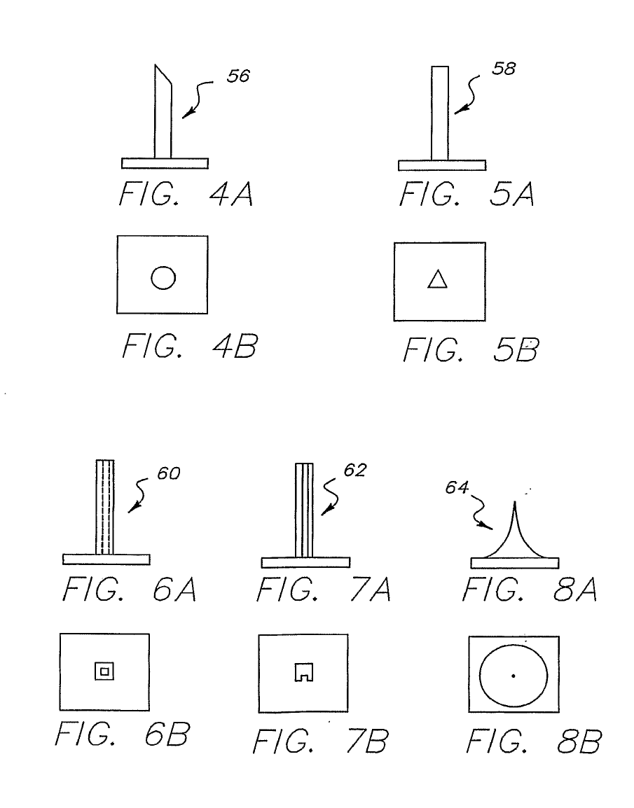

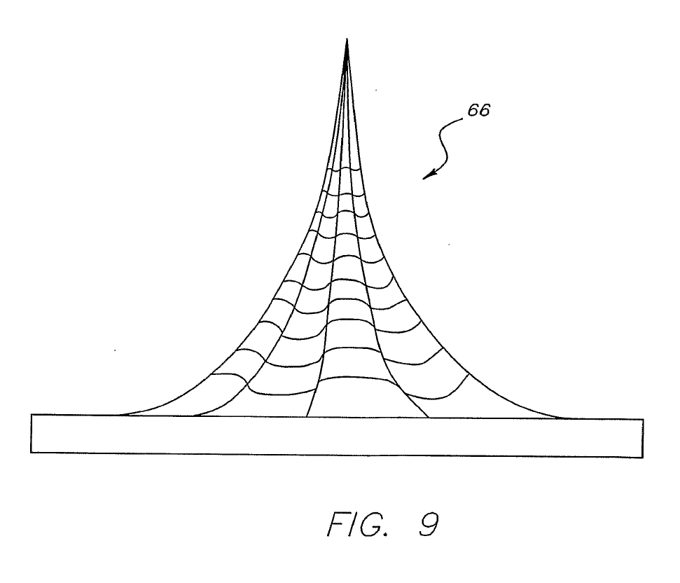

A further embodiment of a microneedle 66 is schematically illustrated in FIG. 9. In this embodiment, the microneedle is corrugated around a generally pointed microneedle structure. Corrugated needle designs like this one may facilitate the entry and exit of the microneedle from the test subject 36 with a reduced amount of force. The corrugations may also provide channels for the allergens to enter a puncture site. Furthermore, the corrugations can also provide higher surface area to enhance the amount of material diffusing.

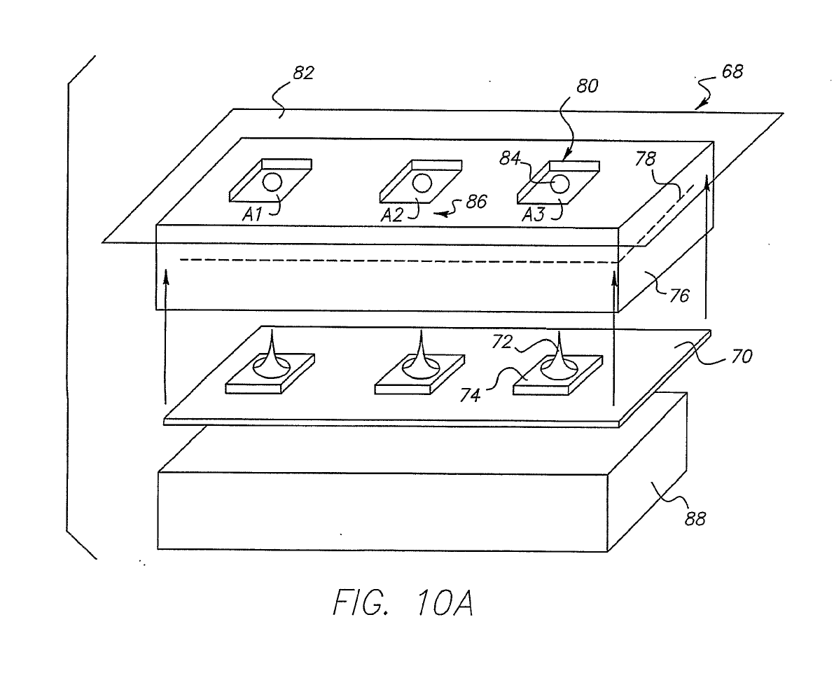

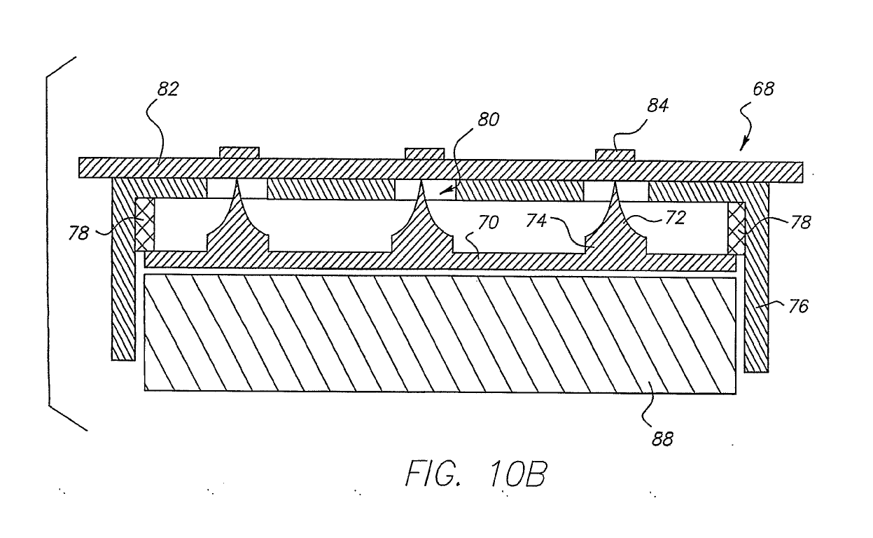

Referring to FIGS. 10A and 10B, another embodiment of a minimally invasive allergy testing system 68 is schematically illustrated in an exploded perspective view and an assembled side cross-sectional view, respectively. The allergy testing system includes a microneedle substrate 70 that supports an array of microneedles 72 with integral mesas 74. The mesas 74 are not absolutely necessary, but in some embodiments, they can provide stability for the microneedles 72 as the microneedles are engaged. (This will be explained in more detail later.) A linear array of microneedles 72 having mesas 74 are illustrated in this embodiment, but other embodiments may have other numbers and types of components in other shapes and configurations. The substrate 70, microneedles 72, and mesas 74 may be manufactured from a number of different materials, such as silicon, glass, metal, or plastic. The microsystem allergy testing device 68 also includes a package assembly 76 which houses the substrate 70, microneedles 72, and mesas 74 such that the top surface of substrate 70 rests against a compressible stop 78 along an inner surface of the package 76. The compressible stop 78 may be permanently compressible, or may be an energy storage device such as a spring. The microneedles 72 are mounted for movement within the package 76 from a position where the upper tips of the microneedles 72 lie within orifices 80, such that they do not protrude significantly outside of the package assembly 76, to a position protruding outside of the package assembly 76.

This embodiment also has a thin sealing film 82 employed over a surface of the package assembly 76. A set of coated allergens 84 associated with each microneedle 72 is coated on the sealing film 82. Prior to testing, the coated allergens 84 may be wet with a solvent so that the coated allergens 84 may be dissolved. In some embodiments, the sealing film 82 may have hydrophobic areas between coated allergens 84 to help prevent intermixing of the dissolved allergens. Such hydrophobic areas may also allow the applied water to selectively wet the allergens 84. The sealing film 82 may also have human readable, barcode, or other identifying marks 86. Such identifying marks have been discussed above with regard to previous embodiments.

This embodiment of an allergy testing system 68 also has a plunger 88. The plunger 88 may be activated by an activation system (not shown), such as manual pressure, mechanical systems, electromechanical systems, piezoelectric, or a micromechanical actuator. Pressure applied to the plunger 88 causes substrate 70 to compress resting stop 78, allowing the microneedles 72 to protrude out of the package assembly 76, pierce through the sealing film 82 and the coated allergens 84 towards the patient's skin (not shown in this figure, but in this embodiment, the test subject would be above the allergy testing system 68 as oriented.) When the plunger 88 is depressed far enough, the microneedles 72 pierce the patient's skin while mesas 74 fit into orifices 80 in package 76 to help increase travel and positional stability of the microneedles 72.

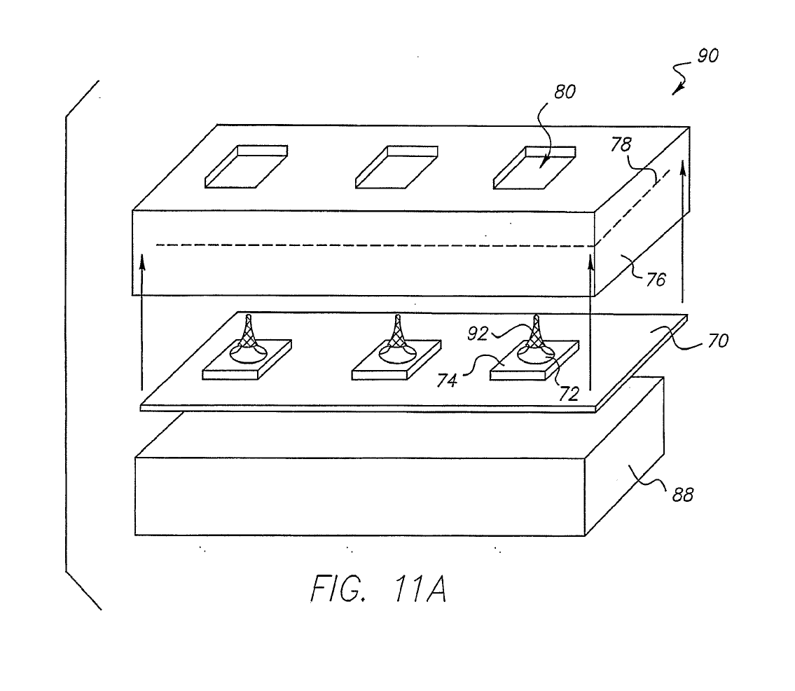

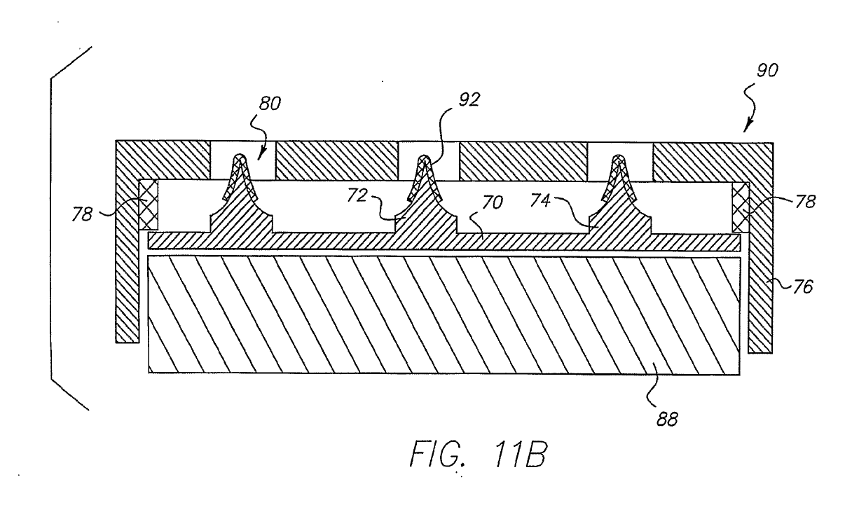

Referring to FIGS. 11A and 11B, another embodiment of a minimally invasive allergy testing system 90 is schematically illustrated in an exploded perspective view and an assembled side cross-sectional view, respectively. The allergy testing system 90 includes a microneedle substrate 70 that supports an array of microneedles 72 with integral mesas 74. The mesas 74 are not absolutely necessary, but in some embodiments, they can provide stability for the microneedles 72 as the microneedles are engaged. A linear array of microneedles having mesas are illustrated in this embodiment, but other embodiments may have other numbers and types of components in other shapes and configurations. The substrate 70, microneedles 72, and mesas 74 may be manufactured from a number of different materials, such as silicon, glass, metal, or plastic. The microneedles 72 are coated with allergens 92, similar to the coated microneedles of FIG. 2A. The microsystem allergy testing device 90 of FIGS. 11A and 11B also includes a package assembly 76 which houses the substrate 70, microneedles 72, and mesas 74 such that the top surface of substrate 70 rests against a compressible stop 78 along an inner surface of the package 76. The compressible stop 78 may be permanently compressible, or may be an energy storage device such as a spring. The microneedles 72 are mounted for movement within the package 76 from a position where the upper tips of the microneedles 72 lie within orifices 80, such that they do not protrude significantly outside of the package assembly 76, to a position protruding outside of the package assembly 76.

This embodiment of an allergy testing system 90 also has a plunger 88. The plunger 88 may be activated by an activation system (not shown), such as manual pressure, mechanical systems, electromechanical systems, piezoelectric, or a micromechanical actuator. Pressure applied to the plunger 88 causes substrate 70 to compress resting stop 78, allowing the microneedles 72 to protrude out of the package assembly 76 towards the patient's skin (not shown in this figure, but in this embodiment, the test subject would be above the allergy testing system 90 as oriented.) When the plunger 88 is depressed far enough, the allergen coated microneedles 72 pierce the patient's skin while mesas 74 fit into orifices 80 in package 76 to help increase travel and positional stability of the microneedles 72. The microneedles 72 may then remain in the skin long enough for the coated allergens 92 to dissolve into the fluids found in the skin, for example, interstitial fluid.

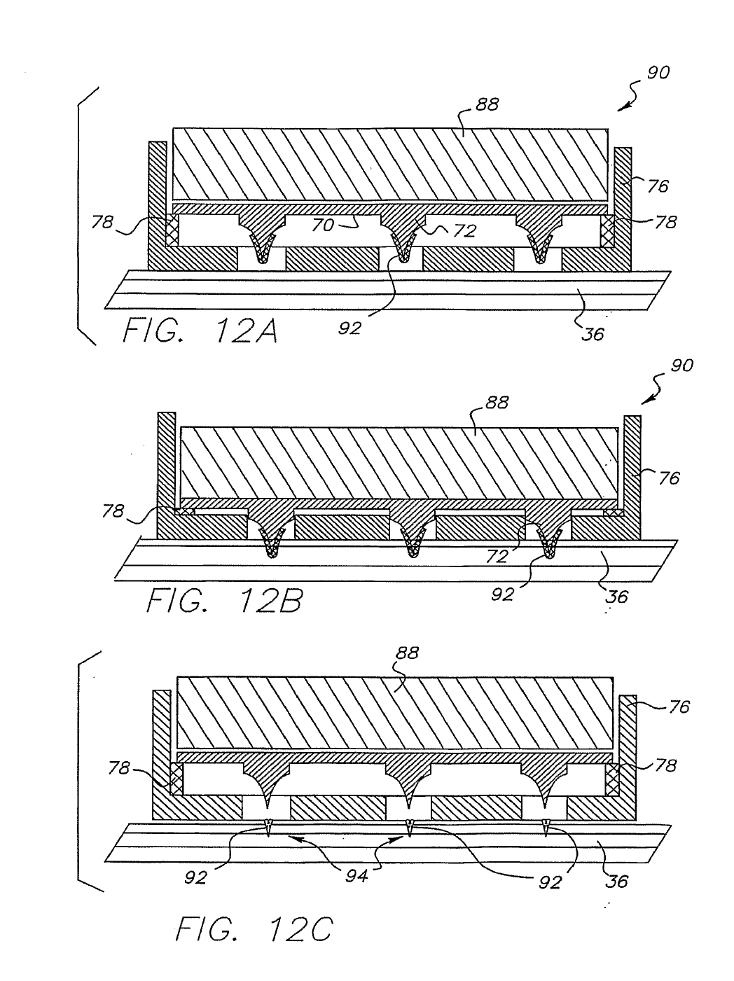

FIGS. 12A-12C schematically illustrate one possible method of applying allergens to a subject using an embodiment of an allergy testing system. In a first step, FIG. 12A, the minimally invasive allergy testing system 90 is placed in contact with a subject's skin 36. Since this embodiment of an allergy testing system 90 does not have a sealing film, the package assembly 76 is the portion of the test system initially in contact with the skin 36. In other systems, the portion initially in contact with the skin 36 might be a sealing film. In FIG. 12A, the microneedle array is in a resting position. In a second step, FIG. 12B, the plunger 88 is activated, in the orientation of FIG. 12B, in a downward direction, causing the substrate 70 to compress the compressible stop 78. This causes the microneedles 72 with coated allergens 92 to puncture the skin 36. In FIG. 12B, the microneedle array is in a penetrating position. After a time sufficient for the allergens 92 to dissolve into the patient's skin 36, the force on the plunger 88 is released, causing it to return to the resting position shown in FIG. 12C. After this procedure, the allergens 92 originally carried by the microneedles 72 are able to diffuse into the subject through the pricked regions 94, thereby potentially initiating allergic reactions.

Examples of various possible needle shapes and geometries have been discussed earlier, and the microneedle shapes illustrated in this example are not intended to be limiting. Furthermore, microneedles of differing lengths on the same substrate 70 may be used. A benefit of providing different length microneedles may be to allow different allergens to reach a targeted skin depth, or to test the same allergen at different depths for studied comparisons.

FIGS. 12D1-12D3 schematically illustrate different embodiments of gathering and analyzing allergy test data after the allergens have been applied by the allergy testing system in FIGS. 12A-12C. FIG. 12D1 schematically illustrates a test subject's skin 36 which has been pricked and injected with allergens 92 by the microneedles 72 of a minimally invasive allergy testing system 90. Here, a schematic human eye 96 is looking for a reaction 98 to a particular allergen 92. A template may be included that matches the applied allergens so as to help identify the corresponding reactions. While the allergy test results may be evaluated using a manual approach such as this, other methods may make it easier to distinguish results given the close spacings between allergy test points possible with microneedles.

FIG. 12D2 schematically illustrates an embodiment of an allergy analysis system 100 which can optionally be used in place of or in conjunction with manual evaluation methods. The allergy analysis system 100 includes an imaging module 102 with imaging optics 104. The imaging optics 104 focuses images of the tested skin area on an image sensor 106. The image sensor 106 is coupled to an image analyzer or processor 108. The image analyzer 108 can analyze the captured images for color, shape, dimension, and location in the test field. Based on this analysis and correlation with what allergen was tested in which location, the analyzer 108 determines reactivity data for each allergen. The reactivity data, as well as the captured images may be stored, displayed, transmitted, and/or printed by the analyzer 108. Optionally, the analyzer may output this data to another processor for storage, display, further analysis, transmission, and/or printing. In the embodiment of FIG. 12D2, the analyzer 108 is directly coupled to the allergy analysis system 100. In other embodiments, the analyzer 108 may be remotely coupled to the analysis system 100 via a wireless or cabled link.

The image analyzer 108 may comprise a central processing unit (CPU) or processor and a memory which are coupled together by a bus or other link, although other numbers and types of components in other configurations and other types of systems, such as an application specific integrated circuit (ASIC) could be used. The processor may execute a program of stored instructions for one or more aspects of the claimed invention, including the method for determining a degree of reaction to one or more allergens as described and illustrated herein. The memory stores these programmed instructions for execution by the processor. A variety of different types of memory storage devices, such as random access memory (RAM) or a read only memory (ROM) in the system or a floppy disk, hard disk, CD ROM, or other computer readable medium which is read from and/or written to by a magnetic, optical, or other reading and/or writing system that is coupled to the processor, can be used for the memory to store these programmed instructions. The image analyzer 108 can be in situ (as illustrated) or remote (for example, separate electronics or a separate computing device) linked by a wired or wireless connection.

In the example of FIG. 12D2, the skin puncture site 110 shows a positive allergy reaction 98, while skin puncture sites 112, 114 show negative allergy reactions. The imaging module 102 should be placed in alignment with the original allergy testing device 90 (from FIGS. 12A-12C) such that the imaging optics 104 creates images of test sites 110, 112, and 114. These test sites 110, 112, 114 are associated with each allergen in correlated locations on image sensor 106. Image patterns associated with each allergen, as imaged by sensor 106, are captured and transmitted to image analyzer 108 for analysis as described above in order to identify color, shape, dimension, allergic reaction, and/or a time rate of change of the allergic response. The determination of a time rate of change in the allergic response may be a great benefit to medical professionals who often do not have the time to personally/physically observe a reaction on a continuous or substantially continuous basis that would let them see how allergic reactions vary over time.

In other embodiments of an allergy testing system which have an allergy imaging analysis system 100, it may be important to determine topographic information when assessing reactivity to an allergen. In such embodiments, an extra set of imaging optics and an extra image sensor may be displaced laterally from the other optical system to obtain stereoscopic, parallax information about a given test location. Parallax information may in turn be used to calculate topographic profiles of test regions.

FIG. 12D3 schematically illustrates an embodiment of a minimally invasive allergy testing system 116 with an integrated imaging and analysis module 102. The microneedle array 118 and plunger 120 operate in similar fashion to the corresponding elements in FIG. 12A, except as described herein. In this embodiment, the array of microneedles 118 and plunger 120 are made of glass or some other transparent material, such as transparent hard plastic, such that imaging module 102 may image the tested regions with the needles in-situ, or even slightly or completely retracted. Preferably, the width of the microneedles 122 relative to their spacing should be fine enough to allow enough imaging area for an adequate diagnosis of the allergic reaction.

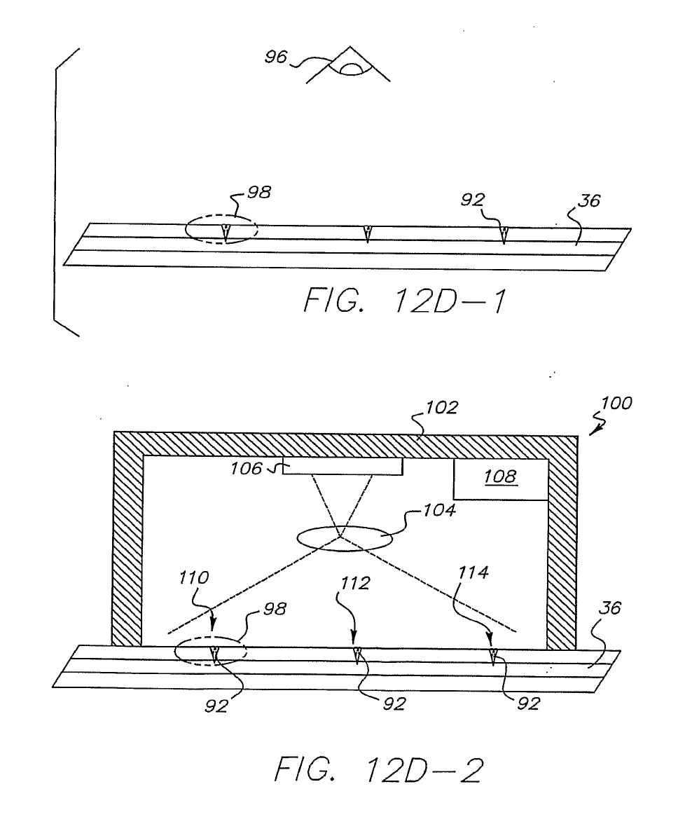

FIG. 13 schematically illustrates a further embodiment of a minimally invasive allergy testing system 124. The allergy testing system 124 is coupled to a band 126 which may be wrapped around a portion of a person's body. In this example, the part of the body illustrated is an arm 128. Of course, it would be apparent to those skilled in the art that attachment band 126 could be compatible with or modified to attach to other portions of the body. Attachment band 126 need not circle completely around and back to itself, although preferred embodiments may have Velcro® attachments which wrap around the body and then connect to themselves. The purpose of the attachment band 126 is to hold a test frame 130 in substantially the same position during a minimally invasive allergy test. The test frame 130 defines an opening in the band 126 through which the skin may be accessed. The test frame 130 also has a first alignment coupling, such as a hinge 132 onto which a package 134 containing a microneedle array having coated allergens may be placed for aligned engagement with the skin in the test frame 130. The test frame 130 has a second alignment coupling, such as a hinge 136 onto which an allergy imaging system 138 may be placed for aligned engagement with the skin in the test frame 130. In some embodiments, the allergen and microneedle package 134 will be engaged with the test frame at different times from the imaging system 138. In other embodiments, such as the transparent embodiments described above, both the allergen and microneedle package 134 and the imaging system 138 may be engaged at the same time. In various embodiments, the alignment coupling does not have to be a hinged connection. It may, instead, be a temporary guide for the manual placement of an otherwise loose portion of the testing system, such as the allergen package 134, or the imaging system 138. In the embodiment of FIG. 13, the analyzer 140 is illustrated as being remotely coupled to the imaging system 138. This remote link may be a physical wire or a radio frequency (RF) or optical wireless link.

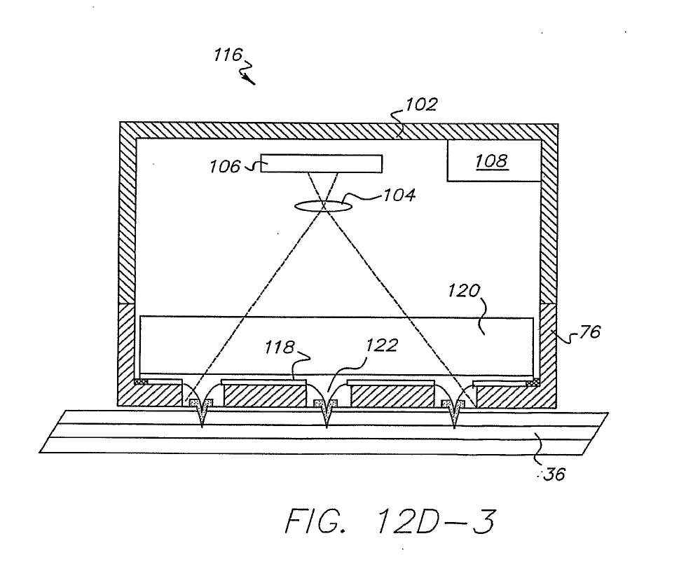

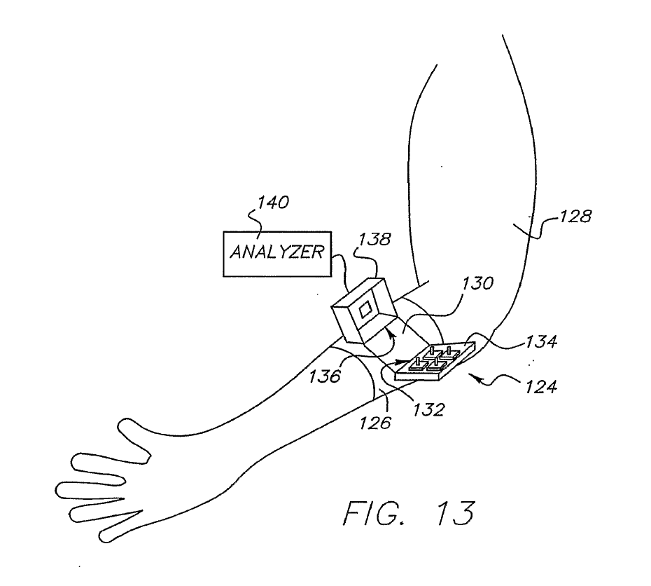

The imaging system 138 from the previous embodiment may be constructed to align-with and/or hold the microneedle package 134 in different ways, depending on the embodiment. For example, FIG. 14A schematically illustrates a side view of an imaging system 142 which holds a replaceable microneedle allergen package 144. The microneedle package 144 may be held in position by guides on the imaging system 142. FIG. 14B schematically illustrates the microneedle allergen package 144 being removed from the imaging system 142 in a substantially linear 146 direction. A microneedle allergen package 144 could be installed by reversing the action illustrated in FIG. 14B. After inserting an allergen package 144, the microneedles may be engaged by the activation system as described in previous embodiments. Then, once the microneedles are allowed to retract from the test-subject's skin 36, the allergen package 144 can be removed from the optical path of the imaging sensor within the imaging system 142 as illustrated in FIG. 14B. In this case, non-transparent or non-optical materials can be used for the microneedle array, such as silicon, metal, opaque plastic, and others. In some cases, it may even be desirable to remove transparent materials away from the optical path in order to reduce requirements for optical flatness, transparency, and cost.

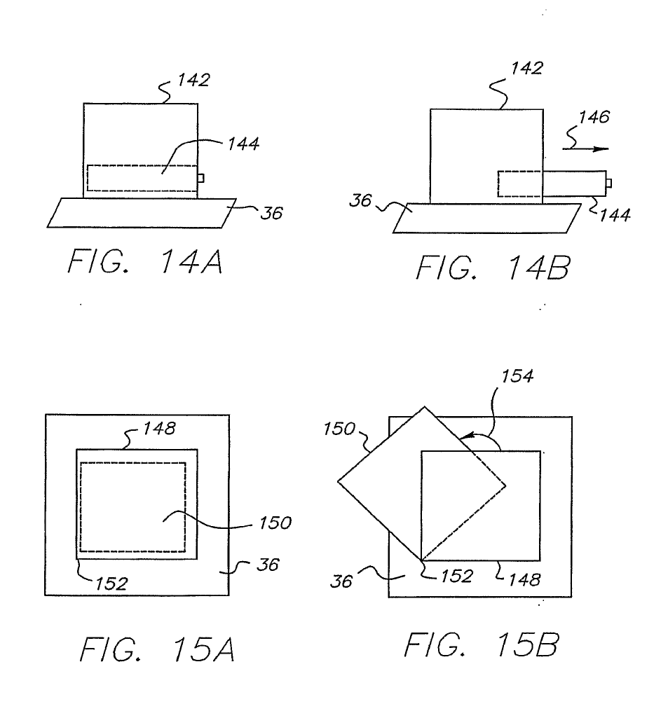

FIG. 15A schematically illustrates a top view of another embodiment of an imaging system 148 which holds a replaceable microneedle allergen package 150. In this embodiment, the microneedle package 150 is held in place by a pivot point 152 and may also be held in position by guides on the imaging system 148. FIG. 15B schematically illustrates the microneedle allergen package 150 being removed from the imaging system 148 in a substantially arcuate 154 direction. A microneedle allergen package 150 could be installed by reversing the action illustrated in FIG. 15B. After inserting an allergen package 150, the microneedles may be engaged by the activation system as described in previous embodiments. Then, once the microneedles are allowed to retract, the allergen package 150 can be removed from the optical path of the imaging sensor within the imaging system 148 as illustrated in FIG. 15B. In this case, non-transparent or non-optical materials can be used for the microneedle array, such as silicon, metal, opaque plastic, and others. In some cases, it may even be desirable to remove transparent materials away from the optical path in order to reduce requirements for optical flatness, transparency, and cost.

Although the descriptions and figures of the embodiments described above show single needle arrays or one dimensional array systems, the claimed invention is easily extendible to two dimensions.

In various embodiments, sheets, thin-films, films, and labels have been described as being used on some embodiments of allergy testing systems and/or on the replaceable allergy testing cartridges, depending on where they were located. All are considered to be equivalents of each other, and for the purposes of the claims, the terms are interchangeable.

Having thus described several embodiments of the claimed invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. Additionally, the recited order of the processing elements or sequences, or the use of numbers, letters, or other designations therefore, is not intended to limit the claimed processes to any order except as may be specified in the claims. Accordingly, the claimed invention is limited only by the following claims and equivalents thereto.

What is claimed is:

1. An allergy testing system, comprising:
a microneedle array;
at least one coated allergen; and
an activation system coupled to the microneedle array such that the at least one coated allergen is moved into contact with a subject as the microneedle array is moved from a resting position to a penetrating position.

2. The allergy testing system of claim 1, wherein the at least one coated allergen is coupled to the microneedle array.

3. The allergy testing system of claim 2, wherein the at least one coated allergen is coupled to the microneedle array with a binder.

4. The allergy testing system of claim 3, wherein the binder is mixed with the at least one coated allergen.

5. The allergy testing system of claim 3, wherein the binder is an interface between the at least one coated allergen and the microneedle array.

6. The allergy testing system of claim 3, wherein the binder is selected from the group consisting of gelatin, glycerol, and biocompatible water soluble polymer.

7. The allergy testing system of claim 2, wherein at least one microneedle in the microneedle array is coupled to the at least one coated allergen.

8. The allergy testing system of claim 2, wherein:
the microneedle array comprises a first side and a second side;
the first side comprises at least one microneedle in the microneedle array; and
the first side further comprises at least one non-microneedle area.

9. The allergy testing system of claim 8, wherein the at least one non-microneedle area is coupled to the at least one coated allergen.

10. The allergy testing system of claim 8, wherein the second side is coupled to the at least one coated allergen.

11. The allergy testing system of claim 10, further comprising a package that at least partially encloses the microneedle array, wherein the microneedle array is moveable within orifices in the package.

12. The allergy testing system of claim 11, wherein the package defines at least one slot to provide access for a solvent to reach the at least one coated allergen coupled to the second side of the microneedle array.

13. The allergy testing system of claim 10, wherein:
the package defines at least one access hole to provide access for a solvent to reach the at least one coated allergen coupled to the second side of the microneedle array; and
the microneedle array comprises at least one channel which fluidically couples the second side of the microneedle array to the first side of the microneedle array.

14. The allergy testing system of claim 13, further comprising at least one plug which may be removeably coupled to the package to block the at least one access hole defined by the package.

15. The allergy testing system of claim 10, wherein the microneedle array comprises at least one channel which fluidically couples the second side of the microneedle array to the first side of the microneedle array.

16. The allergy testing system of claim 1, further comprising a package that at least partially encloses the microneedle array, wherein the microneedle array is moveable relative to the package.

17. The allergy testing system of claim 16, further comprising a sheet coupled to the package, wherein the sheet has a first side which is disposed away from the microneedle array and a second side which is disposed towards the microneedle array.

18. The allergy testing system of claim 17, wherein the at least one coated allergen is coupled to the first side of the sheet.

19. The allergy testing system of claim 18, wherein the first side of the sheet comprises at least one hydrophobic area.

20. The allergy testing system of claim 17, wherein the at least one coated allergen is coupled to the second side of the sheet.

21. The allergy testing system of claim 20, wherein the second side of the sheet comprises at least one hydrophobic area.

22. The allergy testing system of claim 21, wherein:
the package further defines at least one access hole to provide access for a solvent to reach the at least one coated allergen coupled to the second side of the sheet.

23. The allergy testing system of claim 22, further comprising at least one plug which may be removeably coupled to the package to block the at least one access hole defined by the package.

24. The allergy testing system of claim 1, further comprising a sheet which is coupled to the microneedle array, the sheet comprising a first side disposed away from the microneedle array and a second side disposed towards the microneedle array, wherein the at least one coated allergen is coupled to the first side of the sheet.

25. The allergy testing system of claim 24, wherein the first side of the sheet comprises at least one hydrophobic area.

26. The allergy testing system of claim 1, wherein at least one microneedle in the microneedle array comprises a hollow needle.

27. The allergy testing system of claim 1, wherein at least one microneedle in the microneedle array comprises a grooved needle.

28. The allergy testing system of claim 1, wherein at least one microneedle in the microneedle array comprises a corrugated needle.

29. The allergy testing system of claim 1, wherein the microneedle array comprises at least one needle of a first penetration depth and at least one needle of a second penetration depth which is different from the first penetration depth.

30. The allergy testing system of claim 1, wherein the microneedle array comprises at least one needle with a cross-section that is selected from the group consisting of: square, rectangular, triangular, and circular.

31. The allergy testing system of claim 1, wherein the microneedle array comprises at least one needle with a varying cross-section.

32. The allergy testing system of claim 1, wherein the microneedle array comprises silicon.

33. The allergy testing system of claim 1, wherein the microneedle array comprises glass.

34. The allergy testing system of claim 1, wherein the microneedle array comprises quartz.

35. The allergy testing system of claim 1, wherein the microneedle array comprises metal.

36. The allergy testing system of claim 1, wherein the microneedle array comprises plastic.

37. The allergy testing system of claim 1, wherein the microneedle array comprises a substantially transparent material.

38. The allergy testing system of claim 1, further comprising at least one imaging system that captures one or more images of penetration sites by the microneedle array.

39. The allergy testing system of claim 38, wherein the microneedle array is slideably removable from the at least one imaging system to enable the imaging system to capture the one or more images of the penetration sites.

40. The allergy testing system of claim 38 wherein the microneedle array is pivotably removable from the at least one imaging system to enable the imaging system to capture the one or more images of the penetration sites.

41. The allergy testing system of claim 38, wherein the imaging system comprises:
at least one image sensor; and
an imaging optics system which focuses the images of penetration sites on the at least one image sensor.

42. The allergy testing system of claim 41, further comprising:
a second image sensor; and
a second imaging optics system which focuses the images of the penetration sites on the second image sensor; and
wherein the plurality of image sensors are used to determine a topographic profile of an allergic reaction.

43. The allergy testing system of claim 38, wherein the imaging system captures substantially continuous images of the penetration sites.

44. The allergy testing system of claim 38, wherein the imaging system captures images of the penetration sites over time for analysis of a time rate of change of an allergy response.

45. The allergy testing system of claim 1, wherein the activation system comprises an element selected from the group consisting of a mechanical system, an electromechanical system, a piezoelectric system, a micromechanical actuator, and a human actuator.

46. An allergy testing system, comprising:
an attachment band having a test frame, wherein the test frame defines an opening in the attachment band;
a package, for interfacing with the test frame, comprising a microneedle array and coated allergens;
an imaging system for interfacing with the test frame; and
an analyzer coupled to the imaging system.

47. The allergy testing system of claim 46, further comprising:
a first alignment coupling for removeably coupling the package to the test frame; and
a second alignment coupling for removeably coupling the imaging system to the test frame.

48. The allergy testing system of claim 47, wherein the package and the imaging system can be coupled to the test frame at the same time.

49. A method for determining a degree of reaction to one or more allergens by a patient in a minimally invasive manner, comprising:
causing penetration of one or more allergen coated microneedles into a skin of the patient;
capturing one or more images of at least one of the penetrations into the skin;
analyzing at least one of the captured images to assess the degree of reaction to a specific allergen; and
outputting allergic reactivity data for at least one of the allergens.

50. The method of claim 49, further comprising waiting for allergens coating the microneedles to dissolve into fluids found in the skin.

51. The method of claim 49, further comprising wetting the one or more allergen coated microneedles with a solvent to dissolve the coated allergen prior to causing penetration of the one or more allergen coated microneedles into the skin of the patient.

52. The method of claim 49, wherein the solvent comprises water.

53. The method of claim 49, further comprising removing the microneedles before the capturing of images.

54. The method of claim 49, wherein the reactivity data comprises a time rate of change of an allergic response.

55. The method of claim 49, wherein the reactivity data comprises a topographic indicator of an allergic reaction.

56. A method for determining a degree of reaction to one or more allergens by a patient in a minimally invasive manner, comprising:
wetting one or more coated allergens on a sheet to dissolve the coated allergens;
causing penetration of one or more microneedles into a skin of the patient;
exposing each of the penetrations into the skin with dissolved coated allergens;
capturing one or more images of each of the penetrations into the skin;
analyzing at least one of the captured images to assess the degree of reaction to the specific allergen; and
outputting allergic reactivity data for at least one of the allergens.
